(19) United States
(12) Patent Application Publication
(10) Pub. No.: US 2025/0303104 A1
(43) Pub. Date: Oct. 2, 2025

(54) DEVICE FOR INDUCING SLEEP AND IMPROVING BLOOD CIRCULATION

(71) Applicant: KENJI ISHIKAWA, San Mateo, CA (US)

(72) Inventor: KENJI ISHIKAWA, San Mateo, CA (US)

(21) Appl. No.: 18/616,744

(22) Filed: Mar. 26, 2024

(51) Int. Cl. A61M 21/02; A61F 7/00; A61M 21/00

(52) U.S. Cl. CPC A61M 21/02; A61F 7/007; A61F 2007/0011; A61F 2007/0075; A61F 2007/0093; A61M 2021/0027; A61M 2021/0066

(57) ABSTRACT

A device and method for inducing sleep, improving blood circulation, and correcting autonomous imbalances. The device includes a heat conducting plate mounted on top of housing upon which a neck of a user can rest, and the device can apply alternate hot and cold stimuli to the neck. The hot and cold stimuli improve blood circulation and correct the autonomous imbalances. The device includes a drive module and a heat sink to quickly switch from between peak hot and cold temperatures of the alternate hot and cold stimuli.

### FIELD OF INVENTION

The present invention relates to a device for inducing sleep and improving blood circulation, and more particularly, the present invention relates to a device for applying alternate hot and cold cycles to skin for improving blood circulation.

### BACKGROUND

Good sleep is essential for staying healthy. Lack of enough sleep can make a person feel tired all day. Chronic lack of quality sleep can deteriorate health affecting physical and mental well-being and daily functioning. A person may lack good sleep for a variety of reasons or disorders. Insomnia is a most common sleep disorder in which a person is unable to fall asleep and stay asleep. A majority of people today suffer from sleep deprivation. The sleep deprivation in turns causes or aggravates other mental disorders, such as depression and mood disorders.

Several therapies are known in the art of inducing sleep. Medications, such as hypnotics are available, however, the medications themselves have many side effects and are habit-forming. Physical and mental exercises are also known in the art that help in inducing sleep; however, such exercises are complex to practice and often ineffective. The use of some form of sleep aids by people is increasing sleepy.

A need is therefore appreciated for a device and method to induce sleep and improve sleep patterns and overall health.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a device for improving the physical and psychological wellbeing of an individual by inducing healthy sleep and correcting any autonomic imbalances.

It is another object of the present invention to have negligible side effects.

It is still another object of the present invention that the method is not habit-forming.

It is a further object of the present invention to improve overall health.

Still a further object of the present invention is to decrease chronic disease, anxiety, and stress.

It is yet another object of the present invention that the method is easy to use.

In one aspect, disclosed is a device and method of use thereof for applying Alternating Hot and Cold stimuli externally to skin of posterior neck area that increases blood circulation and improves the Autonomic nervous system and stress recovery.

In one aspect, disclosed is a device and a method for inducing sleep, improving blood circulation, and correcting autonomous imbalances. The device includes a housing; a heat conducting plate mounted on a top of the housing, the heat conducting plate is ergonomically designed for maximum contact with posterior neck area for applying the alternate hot and cold stimuli to the posterior neck area. The device further includes a heat sink and a fan for quickly dissipating the heat from the heat conducting plate. A drive module can be operably coupled to the heat conducting plate for heating and cooling the same. The driver module may also be connected to the fan for operating the same.

The drive module is configured to quickly switch between hot and cold states, such as within 20-25 seconds or less. The heat sink and fan help to quickly dissipate the heat from the heat conducting plate.

### BRIEF DESCRIPTION OF THE DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a perspective view of the device, according to an exemplary embodiment of the present invention.

FIG. 2 is a block diagram showing components of the device, according to an exemplary embodiment of the present invention.

FIG. 3A and FIG. 3B show hot and cold cycle stimulus patterns, according to an exemplary embodiment of the present invention.

### DETAILED DESCRIPTION

The subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, reasonably broad scope for claimed or covered subject matter is intended. Among other things, for example, the subject matter may be embodied as apparatus and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an” and “the” are intended to include the plural forms as well, unless the context indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

The following detailed description is described with reference to the drawings, wherein like reference numerals are used to refer to like elements throughout. In the following description, for purposes of explanation, specific details may be set forth in order to provide a thorough understanding of the subject innovation. It may be evident, however, that the claimed subject matter may be practiced without these specific details. In other instances, well-known structures and apparatus are shown in block diagram form in order to facilitate describing the subject innovation. Moreover, the drawings may not be drawn to scale.

Disclosed are a device and a method for transdermal stimulation of the autonomic nerves with instantly alternating hot and cold temperatures stimuli. Such a stimulus can positively affect the sympathetic and parasympathetic nervous response, thereof inducing sleep, improving blood circulation, correcting any autonomous imbalance condition, and improving overall health. Correcting the autonomous imbalance condition can decrease stress and anxiety, resulting in good and natural sleep. Disturbances in the circadian rhythm can be corrected and a person can take a natural quality sleep. The disclosed device can apply instant and alternative hot and cold stimuli cycles on the posterior neck area which results in improved blood circulation and correct any autonomous system imbalances. This in turn results in inducing sleep. Both the improvements in blood circulation and good sleep can lead to improved overall health and wellbeing. Sleep is affected by correcting the autonomous balance system which improves overall good health, and a person can take natural sleep. Sleep disorders and sleep-obsessed conditions can be resolved by regular use of the disclosed device.

Regular use of the disclosed device can activate the autonomic nerves, as well as affect good switching balance between sympathetic and parasympathetic nerves effectively. The disclosed device can target the nerves located in posterior neck/cervical area. In use, a person can lie down on a flat surface and rest their neck on the heat conducting plate of the disclosed device. The alternating hot and cold stimuli can then be applied by the device externally on the posterior neck area.

It is an advantage that the disclosed device can restore a balanced autonomic system. A balanced autonomic system includes Relax/sleeping time (night): Parasympathetic nerve is bigger than Sympathetic nerves, and Active time (day): Parasympathetic nerves are lower than Sympathetic nerves. With regular use of the disclosed device, the balance of the autonomous system and circadian rhythm can be restored, which allows a user to take natural and healthy sleep.

Thus, the disclosed device not only induces sleep, but also affects the whole health conditions, like strengthening the immune system and enteric nerves and increasing blood circulation and oxytocin hormone.

In particular, the alternative hot and cold cycle in short durations helps increasing blood circulation and improves the autonomic nervous system and stress recovery. Hot temperature can dilate the blood vessels while the cold temperature can constrict the blood vessels. Overall result can be improved blood circulation and improvement in the autonomic system.

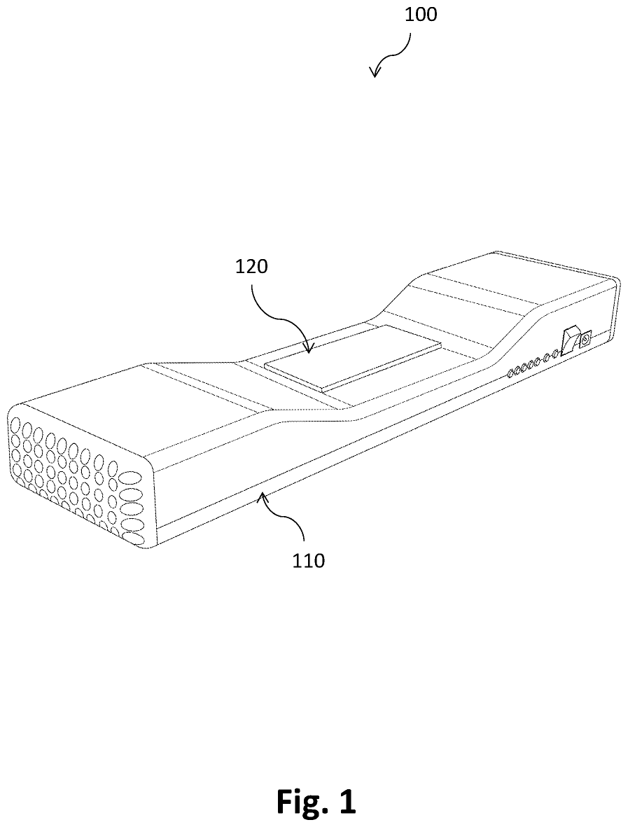

Referring to FIG. 1 shows an exemplary embodiment of the disclosed device 100. Device 100 includes a housing 110 that encases most of the components of the disclosed device and provides an aesthetic appearance to the device. The housing may also protect the device from dirt and external shocks. The housing can be made from an insulative material, such as plastic that prevents any charge build-up and avoids the risk of electric shock. The housing can be thin, narrow, and elongated in dimensions. The housing can have a flat base that allows the device to be stably placed on a flat surface, such as a mattress. The thin profile of the device allows the device to be placed under the neck without any discomfort to the user. The user can lie down on the bed with the device placed on the mattress and the neck of the user rest on the device. It is understood, however, that the device can be placed around the neck while the user is in a sitting position without departing from the scope of the present invention.

On top of the housing can be mounted a heat conducting plate 120. The heat conducting plate comes in contact with the posterior neck area of the user for applying the alternating hot and cold stimuli. So, the heat conducting plate can be ergonomically designed for maximum contact with the posterior neck area of a user using the disclosed device. The heat conducting plate and/or the housing can be suitably and ergonomically designed for comfort of the user and to prevent any injury to the user due to hard edges. Thus, as shown in the drawings, the ergonomic design of the device is critical for the effectiveness and comfort in using the device by the users. The thinner profile of the middle of the device a shown in the drawing may that the neck rests on the heat conducting plate comfortably with maximum heat dissipating efforts. The side portions of the housing are larger and voluminous in size compared to the middle portion for better air flow. For example, four fans, preferably noise-less, can be used for good air circulation. Also, large number of vents are shown for quicker dissipation of heat.

The heat conducting plate can be made from a material that allows uniform dissipation of heat for quickly changing the temperature between hot and cold states, apply the stimuli over the posterior neck area of the user and prevent any localized chills or hot flashes. This rapid change (about 20-25 seconds) between the hot and cold states of the heat conducting plate can be an important element for autonomic nervous stimuli.

The heat conducting plate can be alternatively heated and cooled by a drive module. The drive module can control the stimulus pattern i.e., alternate hot and cold stimuli. The desired pattern can be fed into the drive module and the same can be processed by the drive module. For example, temperatures, duration, and frequency in a form of a configuration file can be configured in the driving module. The driving module may include one or more thermoelectric devices, such as Peltier devices. Thermoelectric devices have an advantage of being silent because of lacking any movable parts. Also, using a heat sink and fan, the heat conducting plate can be quickly cooled from the hot state by dissipating the heat. The drive module can automatically switch between hot and cold peak temperatures in very short durations based on the thermostat temperature. For example, it may take only 20-25 seconds, and more preferably, about 20-21 seconds to switch from the peak hot temperature to the peak cold temperature, and vice versa.

In certain implementations, the drive module includes a control driver chip, Peltier device, thermostats pad for measuring temperature and heat exchange on the heat conducting plates, fuses, buttons to select options and changing settings of the device, and the like. The heat conducing layer can be made of copper, and the like material. Any conducting material for the heat conducting layer is within the scope of the present invention. The device can be powered by a suitable power supply, such as using batteries, home power supply, and the like. The device may include suitable power circuitry, such as an AC to DC adapter for connecting to building power supply. In certain implementation, more than one thermoelectric device can be used for instant change in temperature between peak hot and peak cold temperatures.

The drive module can be configured with different configurations profiles that define the temperatures and their duration. For example, Level 1, starts with letting hot temperature until set the peak hot temperature, and maintain the hot temperature for a fixed duration, and then switch to peak cold temperature and keeping the peak cold temperature for predefined duration. The driver automatically set switching hot/cold temperatures repeatedly 7 times and then switch off the electric power.

Level 2, start letting cold temperature until reaches the peak cold temperature, and maintaining the peak cold temperature for predefined duration, and then switching automatically to the peak cold temperature and keeping the same for predefined duration. This cycle can be repeated seven times and thereafter switch of the power.

Level 3, start letting cold temperature until the peak cold temperature can be achieved and then keeping the cold temperature for desired duration and then automatically switching off the power.

Level 4, start letting hot temperature until peak hot temperature can be achieved, and then keeping the hot temperature for fixed duration, and then automatically switching off the power.

The disclosed device can also include an Arduino module that can control the heating. The drive module can save pre-determined hot and cold temperatures for the stimulation therapy to the neck of the user. As shown in FIG. 1, the device can include buttons for selecting the saved temperature profiles/configuration files as modes.

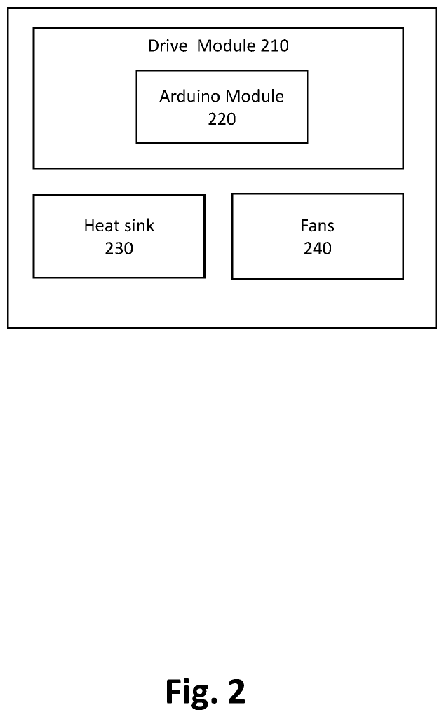

Referring to FIG. 2, shows a block diagram of the disclosed device that includes a drive module 210 to control the disclosed device including setting hot and cold temperatures, operating heatsink and fans, quickly and effectively switching formations, and tuning on/off automatically. The device further includes an Arduino module 220 to control power circuitry including a security fuse system. A specially designed heat sink 230 and a fan 240 to dissipate heat can also be incorporated.

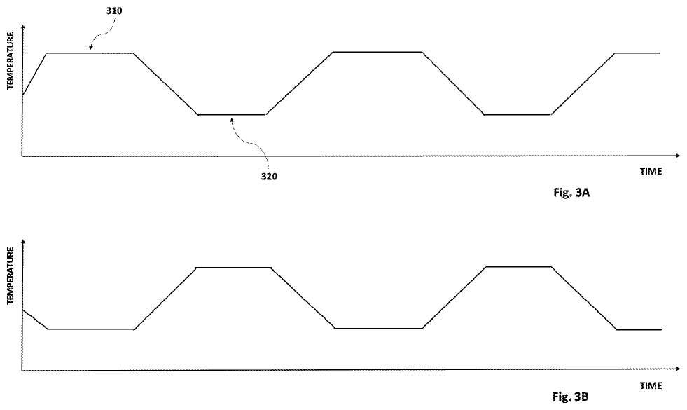

Referring to FIGS. 3A and 3B shows an exemplary therapy cycle for alternative hot and cold stimulus. The disclosed device can heat up to a peak high temperature 310 and cool down to a peak low temperature 320. The peak high temperature, the peak low temperature, and the duration between the peak high temperature and the peak low temperature can be fixed. The peak high temperature and the peak low temperature may not be absolute but can vary within certain limits, such as within two degrees of temperature. One peak high temperature and adjacent peak low temperature can form one cycle and such cycles can be repeated to apply the alternative hot and cold stimulus for a predefined duration. FIG. 3A (mode #1) shows a pattern of alternative cycles of hot and cold stimuli. The duration between the peak low temperature of a cycle and the peak high temperature of an adjacent cycle can also be varied which may be the same or different than the duration (about 20-25 seconds) between the peak high temperature and the peak low temperature of the same cycle. Moreover, the peak high temperatures and the peak low temperatures of different cycles in the same therapy can also be varied. FIG. 3B (mode #2) shows a variation in the cycle in which the peak temperatures are maintained for a predefined duration and this pre-defined duration can be varied without departing from the scope of the present invention.

In certain implementations, the device may also include background music, wherein the device may include an MP3 chip and mini size speaker encased within the housing. The device can play pre-recorded healing music. The user may select a track based on their choices. The music can be turned off automatically when the device is turning off controlled by the drive module.

In certain implementations, the device can be provided with four modes to choose from. Mode #1 can be designed for the wintertime, mode 2 can be designed for summer time), Mode #3 can be designed for a cold day for making warm-up, and mode #4 can be designed for hot days for making cool-down. The device may include a input, such as a button to selected a mode. The selection can be shown using an LED display or simply indicator lights.

Choosing #1 mode (indicates a small green light), Press the start-button to start performing as follows: All performing is controlled by customized/programmed IC tip on the board inside links with the Peltier device. The electric power makes heat-up to the Peltier device and conduct to the plate that would be touched to a user's posterior neck skin (targets to the autonomic nervous on the posterior neck area). After about 20-25 seconds the thermostat sensor connects with the Peltier and heat conducting plate may recognize with 106 degrees and stay on the same temperature for 40 seconds (worm-up to the autonomic nerves). After then change/convert electric + to − on the terminal of the Peltier, conversely the surface of the Peltier becomes COLD temperature with fans blowing the heat air (dissipation of the heat) on the back side. It takes about 20-25 seconds to reach 64 degrees cold temperature on the Peltier and keep the same for 30 seconds cool-down the autonomic nerves. This performing of alternating hot and cold temperatures can be repeated seven times in order to stimuli autonomic nerves.

Mode #2 may be used for the time of summer hot air temperature. Start cooling down to 64 degrees and stays on 40 seconds. After 40 seconds it'll be raising up to 106 degrees and stay on 30 seconds. This process of alternative cold and hot temperature can be repeated seven times.

In Mode #3, Start Hot temperature (106° F., 40° C.) for 180 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds, i.e. the switch Cold (67° F., 20° C.) 30 seconds+switch Hot (106° F., 40° C.) 30 seconds is repeated seven times. The therapy starts with peak hot temperature and ends with peak cold temperature.

In Mode #4, start cold temperature (67° F., 20° C.) for 180 seconds+switch Hot (106° F., 40° C.) 30 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds+switch Cold (67° F., 20° C.) 30 seconds+switch Hot (106° F., 40° C.) 30 seconds (always finish on hot temperature). The cycle Cold (67° F., 20° C.) 30 seconds+switch Hot (106° F., 40° C.) 30 seconds is repeated seven times. The therapy starts with peak cold temperature and ends with peak hot temperature.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A device for transdermal stimulation of the autonomic nerves with alternating hot and cold temperatures stimuli for positively affecting sympathetic and parasympathetic nervous response, inducing sleep, improving blood circulation, and correcting any autonomous imbalance condition, the device comprises:
a housing of an elongated profile having a thinner mid-section and thicker side portions;
a heat conducting plate disposed on a top of the middle-section of the housing; and
a drive module configured for applying the alternating hot and cold temperatures stimuli over a posterior neck area of a user.

2. The device according to claim 1, wherein the drive module comprises one or more thermoelectric units for quickly switching between peak hot temperature and peak cold temperature of the alternating hot and cold temperatures stimuli.

3. The device of claim 1, wherein the drive module is configured to switch between peak hot temperature and peak cold temperature in 20-25 seconds.

4. The device of claim 3, wherein the device further comprises one or more heat sinks and fans for dissipating the heat from heat conducting plate, allowing quicker switching from peak hot temperature to peak cold temperature.

5. The device of claim 3, wherein the drive module is configured to:
warm the heat conducting plate to 106° F. and maintain it at 106° F. for 180 seconds;
after 180 seconds, switching to cold temperature at 67° F. and keeping it at 67° F. for 30 seconds; and
after 30 seconds, switching to hot temperature at 106° F. and keeping it at 106° F. for 30 seconds,
wherein 67° F. for 30 seconds and 106° F. for 30 seconds forms one cycle, wherein the cycle is repeated for seven times.

6. The device of claim 3, wherein the drive module is configured to:
cool the heat conducting plate to 67° F. and maintain it at 67° F. for 180 seconds;
after 180 seconds, switching to hot temperature at 106° F. and keeping it 106° F. at for 30 seconds; and
after 30 seconds, switching to cold temperature at 67° F. and keeping it at 67° F. for 30 seconds,
wherein 106° F. for 30 seconds and 67° F. for 30 seconds forms one cycle, wherein the cycle is repeated for seven times.

7. The device of claim 1, wherein the device further comprises:
an audio chip and a speaker for playing music.
